(19) United States
(12) Patent Application Publication
(10) Pub. No.: US 2005/0153873 A1
(43) Pub. Date: Jul. 14, 2005

(54) Frequency assisted transdermal agent delivery method and system

(76) Inventors: Keith T. Chan, Sunnyvale, CA (US); Michel J.N. Cormier, Mountain View, CA (US); WeiQi Lin, Palo Alto, CA (US)

Correspondence Address: Francis Law Group, 1942 Embarcadero, Oakland, CA, 94606, US

(21) Appl. No.: 10/971,441

(22) Filed: Oct. 21, 2004

Related U.S. Application Data
(60) Provisional application No. 60/535,275, filed on Jan. 9, 2004.

(51) Int. Cl. A61K 38/16; A61K 31/4172; A61M 31/00

(52) U.S. Cl. 514/2; 604/500; 514/397; 514/171

(57) ABSTRACT

An apparatus and method for transdermally delivering a biologically active agent comprising a delivery system having a microprojection member (or system) that includes a plurality of microprojections (or array thereof) that are adapted to pierce through the stratum corneum into the underlying epidermis layer, or epidermis and dermis layers, a formulation containing the biologically active agent and an oscillation inducing device. In one embodiment, the biologically active agent is contained in a biocompatible coating that is applied to the microprojection member. In a further embodiment, the delivery system includes a gel pack having an agent-containing hydrogel formulation that is disposed on the microprojection member after application to the skin of a patient. In an alternative embodiment, the biologically active agent is contained in both the coating and the hydrogel formulation.

### CROSS-REFERENCE TO RELATED APPLICATIONS

This application claims the benefit of U.S. Provisional Application No. 60/535,275, filed Jan. 9, 2004.

### FIELD OF THE PRESENT INVENTION

The present invention relates generally to transdermal agent delivery systems and methods. More particularly, the invention relates to a frequency assisted transdermal agent delivery method and system.

### BACKGROUND OF THE INVENTION

Active agents (or drugs) are most conventionally administered either orally or by injection. Unfortunately, many active agent are completely ineffective or have radically reduced efficacy when orally administered, since they either are not absorbed or are adversely affected before entering the bloodstream and thus do not possess the desired activity. On the other hand, the direct injection of the agent into the bloodstream, while assuring no modification of the agent during administration, is a difficult, inconvenient, painful and uncomfortable procedure which sometimes results in poor patient compliance.

As an alternative, transdermal delivery provides for a method of administering biologically active agents, particularly vaccines, that would otherwise need to be delivered via hypodermic injection, intravenous infusion or orally. Transdermal vaccine delivery offers improvements in both of these areas. Transdermal delivery when compared to oral delivery avoids the harsh environment of the digestive tract, bypasses gastrointestinal drug metabolism, reduces first-pass effects, and avoids the possible deactivation by digestive and liver enzymes. Conversely, the digestive tract is not subjected to the vaccine during transdermal administration.

The word “transdermal”, as used herein, is generic term that refers to delivery of an active agent (e.g., a therapeutic agent, such as a drug or an immunologically active agent, such as a vaccine) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources, such as electricity (e.g., iontophoresis) and ultrasound (e.g., phonophoresis).

However, skin is not only a physical barrier that shields the body from external hazards, but is also an integral part of the immune system. The immune function of the skin arises from a collection of residential cellular and humoral constituents of the viable epidermis and dermis with both innate and acquired immune functions, collectively known as the skin immune system.

One of the most important components of the skin immune system are the Langerhan's cells (LC), which are specialized antigen presenting cells found in the viable epidermis. LC's form a semi-continuous network in the viable epidermis due to the extensive branching of their dendrites between the surrounding cells. The normal function of the LC's is to detect, capture and present antigens to evoke an immune response to invading pathogens. LC's perform his function by internalizing epicutaneous antigens, trafficking to regional skin-draining lymph nodes, and presenting processed antigens to T cells.

The effectiveness of the skin immune system is responsible for the success and safety of vaccination strategies that have been targeted to the skin. Vaccination with a live-attenuated smallpox vaccine by skin scarification has successfully led to global eradication of the deadly small pox disease. Intradermal injection using ⅕ to 1/10 of the standard IM doses of various vaccines has been effective in inducing immune responses with a number of vaccines while a low-dose rabies vaccine has been commercially licensed for intradermal application.

Transdermal delivery offers significant advantages for vaccination, given the function of the skin as an immune organ. Pathogens entering the skin are confronted with a highly organized and diverse population of specialized cells capable of eliminating microorganisms through a variety of mechanisms. Epidermal Langerhans cells are potent antigen-presenting cells. Lymphocytes and dermal macrophages percolate throughout the dermis. Keratinocytes and Langerhans cells express or can be induced to generate a diverse array of immunologically active compounds. Collectively, these cells orchestrate a complex series of events that ultimately control both innate and specific immune responses.

It is further thought that non-replicating antigens (i.e., killed viruses, bacteria, an subunit vaccines) enter the endosomal pathway of antigen presenting cells. The antigens are processed and expressed on the cell surface in association with class II MHC molecules, leading to the activation of CD4⁺ T cells. Experimental evidence indicates that introduction of antigens exogenously induces little or no cell surface antigen expression associated with class I MHC, resulting in ineffective CD8⁺ T activation. Replicating vaccines, on the other hand (e.g., live, attenuated viruses, such as polio and smallpox vaccines) lead to effective humoral and cellular immune responses and are considered the “gold standard” among vaccines. A similar broad immune response spectrum can be achieved by DNA vaccines.

In contrast, polypeptide based vaccines, like subunit vaccines, and killed viral and bacterial vaccines do elicit predominantly a humoral response, as the original antigen presentation occurs via the class II MHC pathway. A method to enable the presentation of these vaccines also via the class I MHC pathway would be of great value, as it would widen the immune response spectrum.

Several reports have suggested that soluble protein antigens can be formulated with surfactants, leading to antigen presentation via the class I pathway and induce antigen-specific class I-restricted CTLs (Raychaudhuri, et al., 1992). Introduction of protein antigen by osmotic lyses of pinosomes has also been demonstrated to lead to a class I antigen-processing pathway (Moore, et al.). Ultrasound techniques have been used to introduce macromolecules into cells in vitro and in vivo, and, particularly, DNA-based therapeutics. Studies with plasmid DNA have clearly demonstrated that the delivery efficiency can be significantly enhanced when ultrasound is employed.

There is, however, no published literature regarding in vivo intracellular ultrasound delivery of protein-based vaccine molecules into skin antigen-presenting cells (APC) that leads to cellular expression of the protein onto class I MHC/HLA presentation molecules in addition to class II MHC/HLA presentation molecules. In particular, there is no mention of the use of a microprojection array in conjunction with ultrasound to achieve this means.

There is also no published literature mentioning the use of a microprojection array in conjunction with ultrasound to achieve in vivo delivery of a DNA vaccine intracellularly and subsequent cellular expression of the protein onto class I MHC/HLA presentation molecules in addition to class II MHC/HLA presentation molecules.

As is well known in the art, the transdermal drug flux is dependent upon the condition of the skin, the size and physical/chemical properties of the drug molecule, and the concentration gradient across the skin. Because of the low permeability of the skin to many drugs, transdermal delivery has had limited applications. For example, in many instances the flux of agents via the traditional passive transdermal routes is too limited to be immunologically effective. This low permeability is attributed primarily to the stratum corneum, the outermost skin layer which consists of flat, dead cells filled with keratin fibers (i.e., keratinocytes) surrounded by lipid bilayers. This highly-ordered structure of the lipid bilayers confers a relatively impermeable character to the stratum corneum.

One common method of increasing the passive transdermal diffusional agent flux involves pre-treating the skin with, or co-delivering with the agent, a skin permeation enhancer. A permeation enhancer, when applied to a body surface through which the agent is delivered, enhances the flux of the agent therethrough. However, the efficacy of these methods in enhancing transdermal protein flux has been limited, at least for the larger proteins, due to their size.

There also have been many techniques and systems developed to mechanically penetrate or disrupt the outermost skin layers thereby creating pathways into the skin in order to enhance the amount of agent being transdermally delivered. Illustrative are skin scarification devices, or scarifiers, which typically provide a plurality of tines or needles that are applied to the skin to scratch or make small cuts in the area of application. The vaccine is applied either topically on the skin, such as disclosed in U.S. Pat. No. 5,487,726, or as a wetted liquid applied to the scarifier tines, such as disclosed in U.S. Pat. Nos. 4,453,926, 4,109,655, and 3,136,314.

Scarifiers have been suggested for intradermal vaccine delivery, in part, because only very small amounts of the vaccine need to be delivered into the skin to be effective in immunizing the patient. Further, the amount of vaccine delivered is not particularly critical since an excess amount also achieves satisfactory immunization.

A major drawback associated with the use of a scarifier to deliver an active agent, such as a vaccine, is the difficulty in determining the transdermal agent flux and the resulting dosage delivered. Also, due to the elastic, deforming and resilient nature of skin to deflect and resist puncturing, the tiny piercing elements often do not uniformly penetrate the skin and/or are wiped free of a liquid coating of an agent upon skin penetration.

Other systems and apparatus that employ tiny skin piercing elements to enhance transdermal agent delivery are disclosed in U.S. Pat. Nos. 5,879,326, 3,814,097, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

The disclosed systems further typically include a reservoir for holding the agent and also a delivery system to transfer the agent from the reservoir through the stratum corneum, such as by hollow tines of the device itself. One example of such a device is disclosed in WO 93/17754, which has a liquid agent reservoir. The reservoir must, however, be pressurized to force the liquid agent through the tiny tubular elements and into the skin. Disadvantages of such devices include the added complication and expense for adding a pressurizable liquid reservoir and complications due to the presence of a pressure-driven delivery system.

As disclosed in U.S. patent application Ser. No. 10/045,842, which is fully incorporated by reference herein, it is possible to have the active agent that is to be delivered coated on the microprojections instead of contained in a physical reservoir. This eliminates the necessity of a separate physical reservoir and developing an agent formulation or composition specifically for the reservoir.

A drawback of such coated microprojection systems is that the maximum amount of delivered active agent is limited, since the ability of the microprojections (and arrays thereof) to penetrate the stratum corneum is reduced as the coating thickness increases. Further, to effectively penetrate the stratum corneum with microprojections having a thick coating disposed thereon, the impact energy of the applicator must be increased, which causes intolerable sensations upon impact. A further drawback is that they are limited to a bolus-type agent delivery profile.

Active transport systems have also been employed to enhance agent flux through the stratum corneum. One such system for transdermal agent delivery is referred to as “electrotransport”. The noted system employs an electric potential, which results in the application of electric current is aid in the transport of the agent through the stratum corneum.

A further active transport system, commonly referred to as “phonophoresis”, employs ultrasound (i.e., sound waves) to aid in the transport of the agent through the stratum corneum. Illustrative are the systems disclosed in U.S. Pat. No. 5,733,572 and Pat. Pub. No. 2002/0099356 A1.

In U.S. Pat. No. 5,733,572, an active system is disclosed that includes gas-filled microspheres as topical and subcutaneous delivery vehicles. The microspheres are made to encapsulate agents and are injected or otherwise administered to a patient. Ultrasonic energy is then used to rupture the microspheres to release the agent.

The ultrasound applied to the microspheres has a frequency in the range of 0.5 MHz and 10 MHz. This range of frequencies has, however, been shown to be of limited use in producing cavitation effects in skin cells, which are much larger than the size of typical microspheres.

In Pat. Pub. No. 2002/0099356 A1, a further active system is disclosed. The noted system includes a “microneedle array” that utilizes sonic energy to deliver or extract biomolecules through membranes. A major drawback of the noted system is thus the use of microneedles the deliver the active agent. The '356 reference further does not teach or suggest the delivery of a vaccine or any other biologically active agent via coated microprojections.

In Pat Pub. No. 2003/0083645 A1, another active system is disclosed. The noted system similarly employs microneedles to deliver the active agent. In contrast to the aforementioned ultrasound systems, the '645 system employs an oscillator system that is adapted to vibrate the microneedles to enhance the penetration of the microneedles into the skin.

As is well known in the art, there are numerous disadvantages and drawbacks associated with microneedles. Among the drawbacks are the microneedle system complexity and the necessity of additional components and/or systems, such as a reservoir, pump, valves, actuators, etc.

It would therefore be desirable to provide a frequency assisted agent delivery system that employs microprojections and arrays thereof having a biocompatible coating that includes the biologically active agent that is to be delivered.

It is therefore an object of the present invention to provide a frequency assisted transdermal agent delivery method and system that substantially reduces or eliminates the aforementioned drawbacks and disadvantages associated with prior art agent delivery systems.

It is another object of the present invention to provide a frequency assisted transdermal agent delivery method and system that includes microprojections coated with a biocompatible coating that includes at least one biologically active agent.

It is a further object of the present invention to provide a frequency assisted transdermal agent delivery method and system that includes a hydrogel reservoir of at least one biologically active agent for delivery via microprojections.

It is yet another object of the present invention to provide frequency assisted transdermal agent delivery method and system that increases cellular uptake of DNA and conventional vaccines.

### SUMMARY OF THE INVENTION

In accordance with the above objects and those that will be mentioned and will become apparent below, the delivery system for transdermally delivering a biologically active agent to a subject comprises a microprojection member having a plurality of microprojections that are adapted to pierce through the stratum corneum into the underlying epidermis layer, or epidermis and dermis layers, a formulation of the biologically active agent and an oscillation inducing device that is adapted to cooperate with the microprojection member to produce high frequency oscillations.

In a preferred embodiment of the invention, the oscillation inducing device produces substantially uniaxial oscillations of the microprojections in the microprojection member in the range of approximately 10-400 μm.

Alternatively, the oscillation inducing device is adapted to produce substantially transversal oscillations of the microprojections in the microprojection member.

In another embodiment of the invention, the oscillation inducing device is adapted to produce substantially circular oscillations of the microprojections in the microprojection member.

In a preferred embodiment of the invention, the oscillation inducing device provides high frequency vibrations in the range of 200 Hz-100 kHz.

In an additional embodiment, the system further includes an ultrasonic device to enhance transdermal delivery of the biologically active agent. Preferably, the ultrasonic device provides sound waves having a frequency in the range of approximately 20 kHz-10 MHz.

In one embodiment of the invention, the microprojection member has a microprojection density of at least approximately 10 microprojections/cm², more preferably, in the range of at least approximately 200-2000 microprojections/cm².

In one embodiment, the microprojection member is constructed out of stainless steel, titanium, nickel titanium alloys, or similar biocompatible materials, such as polymeric materials.

In an alternative embodiment, the microprojection member is constructed out of a non-conductive material, such as a polymer. Alternatively, the microprojection member can be coated with a non-conductive material, such as Parylene®.

In one embodiment of the invention, the biologically active agent comprises a vaccine, an antigenic agent or an immunologically active agent. The vaccine can include viruses and bacteria, protein-based vaccines, polysaccharide-based vaccine, and nucleic acid-based vaccines.

Suitable antigenic agents include, without limitation, antigens in the form of proteins, polysaccharide conjugates, oligosaccharides, and lipoproteins. These subunit vaccines in include Bordetella pertussis (recombinant PT accince—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-1 1, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diptheriae, group A streptococcus, legionella pneumophila, neisseria meningitis, pseudomonas aeruginosa, streptococcus pneumoniae, treponema pallidum, and vibrio cholerae, and mixtures thereof.

Additional commercially available vaccines, which contain antigenic agents, include, without limitation, flu vaccines, Lyme disease vaccine, rabies vaccine, measles vaccine, mumps vaccine, chicken pox vaccine, small pox vaccine, hepatitis vaccine, pertussis vaccine, and diptheria vaccine.

Vaccines comprising nucleic acids include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. In addition, in certain embodiments of the invention, the nucleic acid can be coupled with a proteinaceous agent or can include one or more chemical modifications, such as, for example, phosphorothioate moieties. The encoding sequence of the nucleic acid comprises the sequence of the antigen against which the immune response is desired. In addition, in the case of DNA, promoter and polyadenylation sequences are also incorporated in the vaccine construct. The antigen that can be encoded include all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants which, together with the vaccine antigen, can comprise the vaccine include aluminum phosphate gel; aluminum hydroxide; algal glucan: b-glucan; cholera toxin B subunit; CRL1005: ABA block polymer with mean values of x=8 and y=205; gamma inulin: linear (unbranched) β-D(2->1)polyfructofuranoxyl-a-D-glucose; Gerbu adjuvant: N-acetylglucosamine-(b 1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher®: N-acetylglucoaminyl-N-acetylmuramyl-L-Ala-D-isoGlu-L-Ala-glycerol dipalmitate; MTP-PE liposomes: C59H108N6O19PNa-3H20 (MTP); Murametide: Nac-Mur-L-Ala-D-Gln-OCH3; Pleuran: b-glucan; QS-21; S-28463: 4-amino-a, a-dimethyl-1H-imidazo[4,5-c]quinoline-1-ethanol; sclavo peptide: VQGEESNDK·HCl (IL-1b 163-171 peptide); and threonyl-MDP (Termurtide®): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used.

Alternatively, the formulation comprises other biologically active agents. Suitable active agents include, without limitation, leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotropin (ACTH), ACTH analogs such as ACTH (1-24), calcitonin, vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N-[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, BNP, VEGF, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and mixtures thereof.

In one embodiment of the invention, the formulation comprises a biocompatible coating that is disposed on at least the microprojections.

The coating formulations applied to the microprojection member to form solid coatings can comprise aqueous and non-aqueous formulations having at least one biologically active agent, which can be dissolved within a biocompatible carrier or suspended within the carrier.

In one embodiment of the invention, the coating formulations include at least one surfactant, which can be zwitterionic, amphoteric, cationic, anionic, or nonionic. Examples of suitable surfactants include sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives, such as sorbitan laurate, and alkoxylated alcohols such as laureth-4.

In one embodiment of the invention, the concentration of the surfactant is in the range of approximately 0.001-2 wt. % of the coating solution formulation.

In a further embodiment of the invention, the coating formulations include at least one polymeric material or polymer that has amphiphilic properties, which can comprise, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxyethylcellulose (EHEC), as well as pluronics.

In one embodiment of the invention, the concentration of the polymer presenting amphiphilic properties is preferably in the range of approximately 0.01-20 wt. %, more preferably, in the range of approximately 0.03-10 wt. % of the coating.

In another embodiment, the coating formulations include a hydrophilic polymer selected from the following group: poly(vinyl alcohol), poly(ethylene oxide), poly(2-hydroxyethylmethacrylate), poly(n-vinyl pyrolidone), polyethylene glycol and mixtures thereof, and like polymers.

In a preferred embodiment, the concentration of the hydrophilic polymer in the coating formulation is in the range of approximately 0.01-20 wt. %, more preferably, in the range of approximately 0.03-10 wt. % of the coating formulation.

In another embodiment of the invention, the coating formulations include a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose and stachyose.

Preferably, the concentration of the biocompatible carrier in the coating formulation is in the range of approximately 2-70 wt. %, more preferably, in the range of approximately 5-50 wt. % of the coating formulation.

In a further embodiment, the coating formulations include a stabilizing agent, which can comprise, without limitation, a non-reducing sugar, a polysaccharide, a reducing sugar or a DNase inhibitor.

In another embodiment, the coating formulations include a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating.

In yet another embodiment of the invention, the coating formulations include at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In a further embodiment of the invention, the coating formulation includes at least one antioxidant, which can be sequestering such as sodium citrate, citric acid, EDTA (ethylene-dinitrilo-tetraacetic acid) or free radical scavengers such as ascorbic acid, methionine, sodium ascorbate, and the like. Presently preferred antioxidants include EDTA and methionine.

In certain embodiments of the invention, the viscosity of the coating formulation is enhanced by adding low volatility counterions. In one embodiment, the agent has a positive charge at the formulation pH and the viscosity-enhancing counterion comprises an acid having at least two acidic pKas. Suitable acids include maleic acid, malic acid, malonic acid, tartaric acid, adipic acid, citraconic acid, fumaric acid, glutaric acid, itaconic acid, meglutol, mesaconic acid, succinic acid, citramalic acid, tartronic acid, citric acid, tricarballylic acid, ethylenediaminetetraacetic acid, aspartic acid, glutamic acid, carbonic acid, sulfuric acid, and phosphoric acid.

Another preferred embodiment is directed to a viscosity-enhancing mixture of counterions wherein the agent has a positive charge at the formulation pH and at least one of the counterion is an acid having at least two acidic pKas. The other counterion is an acid with one or more pKas. Examples of suitable acids include hydrochloric acid, hydrobromic acid, nitric acid, sulfuric acid, maleic acid, phosphoric acid, benzene sulfonic acid, methane sulfonic acid, citric acid, succinic acid, glycolic acid, gluconic acid, glucuronic acid, lactic acid, malic acid, pyruvic acid, tartaric acid, tartronic acid, fumaric acid, acetic acid, propionic acid, pentanoic acid, carbonic acid, malonic acid, adipic acid, citraconic acid, levulinic acid, glutaric acid, itaconic acid, meglutol, mesaconic acid, citramalic acid, citric acid, aspartic acid, glutamic acid, tricarballylic acid and ethylenediaminetetraacetic acid.

Generally, in the noted embodiments of the invention, the amount of counterion should neutralize the charge of the antigenic agent. In such embodiments, the counterion or the mixture of counterion is present in amounts necessary to neutralize the charge present on the agent at the pH of the formulation. Excess of counterion (as the free acid or as a salt) can be added to the formulation in order to control pH and to provide adequate buffering capacity.

In another preferred embodiment, the agent has a positive charge and the counterion is a viscosity-enhancing mixture of counterions chosen from the group of citric acid, tartaric acid, malic acid, hydrochloric acid, glycolic acid, and acetic acid. Preferably, counterions are added to the formulation to achieve a viscosity in the range of about 20-200 cp.

In a preferred embodiment, the viscosity-enhancing counterion is an acidic counterion such as a low volatility weak acid. Low volatility weak acid counterions present at least one acidic pKa and a melting point higher than about 50° C. or a boiling point higher than about 170° C. at Pₐₜₘ. Examples of such acids include citric acid, succinic acid, glycolic acid, gluconic acid, glucuronic acid, lactic acid, malic acid, pyruvic acid, tartaric acid, tartronic acid, and fumaric acid.

In another preferred embodiment the counterion is a strong acid. Strong acids can be defined as presenting at least one pKa lower than about 2. Examples of such acids include hydrochloric acid, hydrobromic acid, nitric acid, sulfonic acid, sulfuric acid, maleic acid, phosphoric acid, benzene sulfonic acid and methane sulfonic acid.

Another preferred embodiment is directed to a mixture of counterions wherein at least one of the counterion is a strong acid and at least one of the counterion is a low volatility weak acid.

Another preferred embodiment is directed to a mixture of counterions wherein at least one of the counterions is a strong acid and at least one of the counterion is a weak acid with high volatility. Volatile weak acid counterions present at least one pKa higher than about 2 and a melting point lower than about 50° C. or a boiling point lower than about 170° C. at Pₐₜₘ. Examples of such acids include acetic acid, propionic acid, pentanoic acid and the like.

Preferably, the acidic counterion is present in amounts necessary to neutralize the positive charge present on the antigenic agent at the pH of the formulation. Excess of counterion (as the free acid or as a salt) can be added to the formulation in order to control pH and to provide adequate buffering capacity.

In yet other embodiments of the invention, particularly where the antigenic agent has a negative charge, the coating formulation further comprises a low volatility basic counter ion.

In a preferred embodiment, the coating formulation comprises a low volatility weak base counterion. Low volatility weak bases present at least one basic pKa and a melting point higher than about 50° C. or a boiling point higher than about 170° C. at Pₐₜₘ. Examples of such bases include monoethanolomine, diethanolamine, triethanolamine, tromethamine, methylglucamine, and glucosamine.

In another embodiment, the low volatility counterion comprises a basic zwitterions presenting at least one acidic pKa, and at least two basic pKa's, wherein the number of basic pKa's is greater than the number of acidic pkA's. Examples of such compounds include histidine, lysine, and arginine.

In yet other embodiments, the low volatility counterion comprises a strong base presenting at least one pKa higher than about 12. Examples of such bases include sodium hydroxide, potassium hydroxide, calcium hydroxide, and magnesium hydroxide.

Other preferred embodiments comprise a mixture of basic counterions comprising a strong base and a weak base with low volatility. Alternatively, suitable counterions include a strong base and a weak base with high volatility. High volatility bases present at least one basic pKa lower than about 12 and a melting point lower than about 50° C. or a boiling point lower than about 170° C. at Pₐₜₘ. Examples of such bases include ammonia and morpholine.

Preferably, the basic counterion is present in amounts necessary to neutralize the negative charge present on the antigenic agent at the pH of the formulation. Excess of counterion (as the free base or as a salt) can be added to the formulation in order to control pH and to provide adequate buffering capacity.

Preferably, the coating formulations have a viscosity less than approximately 500 centipoise and greater than 3 centipoise.

In one embodiment of the invention, the coating thickness is less than 25 microns, more preferably, less than 10 microns as measured from the microprojection surface.

In another aspect of the invention, the formulation comprises a hydrogel which can be incorporated into a gel pack.

Correspondingly, in certain embodiments of the invention, the hydrogel formulations contain at least one biologically active agent. Preferably, the agent comprises one of the aforementioned vaccines, including, without limitation, viruses and bacteria, protein-based vaccines, polysaccharide-based vaccine, and nucleic acid-based vaccines or one of the other aforementioned biologically active agents.

The hydrogel formulations preferably comprise water-based hydrogels having macromolecular polymeric networks.

In a preferred embodiment of the invention, the polymer network comprises, without limitation, hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics.

The hydrogel formulations preferably include one surfactant, which can be zwitterionic, amphoteric, cationic, anionic, or nonionic.

In one embodiment of the invention, the surfactant can comprise sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives such as sorbitan laurate, and alkoxylated alcohols such as laureth-4.

In another embodiment, the hydrogel formulations include polymeric materials or polymers having amphiphilic properties, which can comprise, without limitation, cellulose derivatives, such as hydroxyethylcellulose (HEC), hydroxypropyl-methylcellulose (HPMC), hydroxypropycellulose (HPC), methylcellulose (MC), hydroxyethylmethylcellulose (HEMC), or ethylhydroxyethylcellulose (EHEC), as well as pluronics.

In a further embodiment of the invention, the hydrogel formulations contain at least one pathway patency modulator, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, and EDTA.

In yet another embodiment of the invention, the hydrogel formulations include at least one vasoconstrictor, which can comprise, without limitation, epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline, and the mixtures thereof.

In a further aspect of the gel pack embodiments, the biologically active agent can be contained in a hydrogel formulation in the gel pack and in a biocompatible coating applied to the microprojection member.

In accordance with one embodiment of the invention, the method for delivering a biologically active agent (contained in the hydrogel formulation or contained in the biocompatible coating on the microprojection member or both) comprises applying the microprojection member to a mammal's skin, preferably via an actuator, and operating the oscillation inducing device to facilitate penetration of the microprojections through the stratum corneum. Preferably, the oscillation inducing device produces high frequency vibrations in the range of approximately 200 Hz-100 kHz.

In certain embodiments, the oscillation inducing device is incorporated into the microprojection member. Alternatively, the oscillation inducing device comprises a separate device that is placed on the microprojection member after the microprojection member is applied to the mammal's skin.

The methods of the invention comprise producing substantially uniaxial oscillations, substantially transversal or substantially circular oscillations in the microprojections with the oscillation inducing device to facilitate penetration of the microprojections through the stratum corneum.

A further embodiment of the invention comprises providing an ultrasonic device and transmitting energy from said ultrasonic device after application of the microprojection member to facilitate delivery of the biologically active agent. Preferably, this comprises transmitting energy from the ultrasonic device in the range of approximately 20 kHz to 10 MHz.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1A is a schematic illustration of one embodiment of a oscillation inducing device for transdermally delivering a biologically active agent, according to the invention;

FIG. 1B is a schematic illustration of one embodiment of a oscillation inducing device having a microprojection member for transdermally delivering a biologically active agent, according to the invention;

FIG. 2 is a perspective view of a portion of one example of a microprojection member;

FIG. 3 is a perspective view of the microprojection member shown in FIG. 2 having a coating deposited on the microprojections, according to the invention;

FIG. 3A is a cross-sectional view of a single microprojection taken along line 2A-2A in FIG. 3, according to the invention;

FIG. 4 is a side sectional view of a microprojection member having an adhesive backing;

FIG. 5 is a side sectional view of a retainer having a microprojection member disposed therein;

FIG. 6 is a perspective view of the retainer shown in FIG. 5;

FIG. 7 is an exploded perspective view of one embodiment of a gel pack of a microprojection system;

FIG. 8 is an exploded perspective view of one embodiment of a microprojection assembly that is employed in conjunction with the gel pack shown in FIG. 7; and

FIG. 9 is a perspective view of another embodiment of a microprojection system.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a microprojection” includes two or more such microprojections and the like.

### Definitions

The term “transdermal”, as used herein, means the delivery of an agent into and/or through the skin for local or systemic therapy.

The term “transdermal flux”, as used herein, means the rate of transdermal delivery.

The term “co-delivering”, as used herein, means that a supplemental agent(s) is administered transdermally either before the agent is delivered, before and during transdermal flux of the agent, during transdermal flux of the agent, during and after transdermal flux of the agent, and/or after transdermal flux of the agent. Additionally, two or more biologically active agents may be formulated in the coatings and/or hydrogel formulation, resulting in co-delivery of the biologically active agents.

The term “biologically active agent”, as used herein, refers to a composition of matter or mixture containing an active agent or drug, which is pharmacologically effective when administered in a therapeutically effective amount. Examples of such active agents include, without limitation, small molecular weight compounds, polypeptides, proteins, oligonucleotides, nucleic acids and polysaccharides.

Further examples of “biologically active agents” include, without limitation, the leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotropin (ACTH), ACTH analogs such as ACTH (1-24), calcitonin, vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N-[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, BNP, VEGF, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and mixtures thereof.

The term “biologically active agent”, as used herein, also refers to a composition of matter or mixture containing a “vaccine” or other immunologically active agent, such as an antigen, which is capable of triggering a beneficial immune response when administered in an immunologically effective amount. Examples of such agents include, without limitation, viruses and bacteria, protein-based vaccines, polysaccharide-based vaccine, and nucleic acid-based vaccines.

Suitable antigenic agents that can be used in the present invention include, without limitation, antigens in the form of proteins, polysaccharide conjugates, oligosaccharides, and lipoproteins. These subunit vaccines in include Bordetella pertussis (recombinant PT accince—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diptheriae, group A streptococcus, legionella pneumophila, neisseria meningitis, pseudomonas aeruginosa, streptococcus pneumoniae, treponema pallidum, and vibrio cholerae, and mixtures thereof.

A number of commercially available vaccines, which contain antigenic agents also have utility with the present invention including, without limitation, flu vaccines, Lyme disease vaccine, rabies vaccine, measles vaccine, mumps vaccine, chicken pox vaccine, small pox vaccine, hepatitis vaccine, pertussis vaccine, and diptheria vaccine.

Vaccines comprising nucleic acids that can be delivered according to the methods of the invention, include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. In addition, in certain embodiments of the invention, the nucleic acid can be coupled with a proteinaceous agent or can include one or more chemical modifications, such as, for example, phosphorothioate moieties. The encoding sequence of the nucleic acid comprises the sequence of the antigen against which the immune response is desired. In addition, in the case of DNA, promoter and polyadenylation sequences are also incorporated in the vaccine construct. The antigen that can be encoded include all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants which, together with the vaccine antigen, can comprise the vaccine include aluminum phosphate gel; aluminum hydroxide; algal glucan: b-glucan; cholera toxin B subunit; CRL1005: ABA block polymer with mean values of x=8 and y=205; gamma inulin: linear (unbranched) β-D(2->1) polyfructofuranoxyl-a-D-glucose; Gerbu adjuvant: N-acetylglucosamine-(b 1-4)—N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C59H108N6O19PNa-3H20 (MTP); Murametide: Nac-Mur-L-Ala-D-Gln-OCH3; Pleuran: b-glucan; QS-21; S-28463: 4-amino-a, a-dimethyl-1H-imidazo[4,5-c]quinoline-1-ethanol; sclavo peptide: VQGEESNDK·HCl (IL-1b 163-171 peptide); and threonyl-MDP (Termurtide™: N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL 10, gamma interferon, and NF kappa B regulatory signaling proteins can be used.

The noted biologically active agents can also be in various forms, such as free bases, acids, charged or uncharged molecules, components of molecular complexes or pharmaceutically acceptable salts. Further, simple derivatives of the active agents (such as ethers, esters, amides, etc.), which are easily hydrolyzed at body pH, enzymes, etc., can be employed.

It is to be understood that more than one biologically active agent can be incorporated into the agent source, reservoirs, and/or coatings of this invention, and that the use of the term “active agent” in no way excludes the use of two or more such active agents or drugs.

The term “biologically effective amount” or “biologically effective rate”, as used herein, means the biologically active agent is an immunologically active agent and refers to the amount or rate of the immunologically active agent needed to stimulate or initiate the desired immunologic, often beneficial result. The amount of the immunologically active agent employed in the hydrogel formulations and coatings of the invention will be that amount necessary to deliver an amount of the active agent needed to achieve the desired immunological result. In practice, this will vary widely depending upon the particular immunologically active agent being delivered, the site of delivery, and the dissolution and release kinetics for delivery of the active agent into skin tissues.

The term “microprojections”, as used herein, refers to piercing elements which are adapted to pierce or cut through the stratum corneum into the underlying epidermis layer, or epidermis and dermis layers, of the skin of a living animal, particularly a mammal and more particularly a human.

In one embodiment of the invention, the piercing elements have a projection length less than 1000 microns. In a further embodiment, the piercing elements have a projection length of less than 500 microns, more preferably, less than 250 microns. The microprojections typically have a width and thickness of about 5 to 50 microns. The microprojections may be formed in different shapes, such as needles, hollow needles, blades, pins, punches, and combinations thereof.

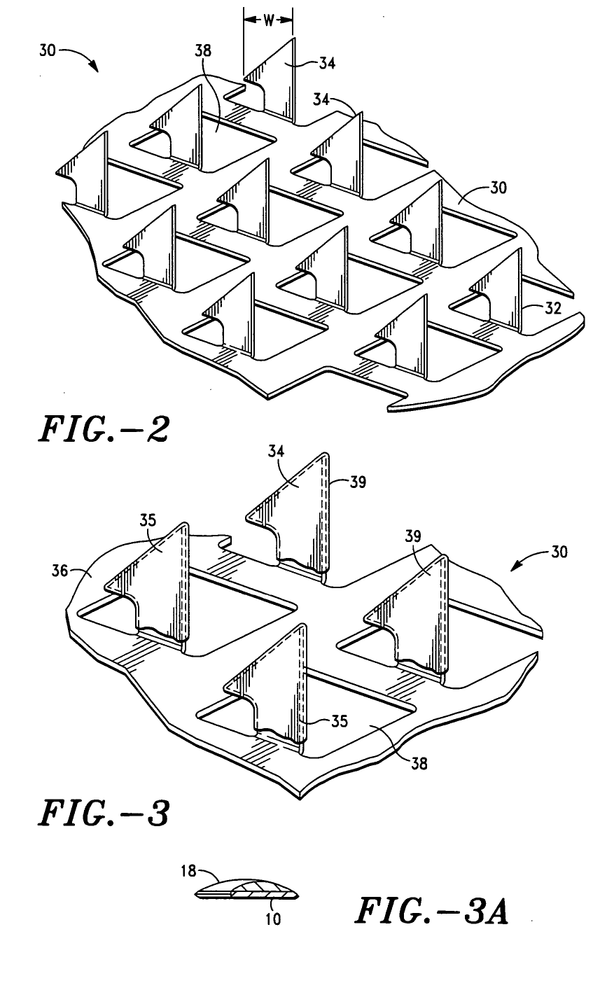

The term “microprojection member”, as used herein, generally connotes a microprojection array comprising a plurality of microprojections arranged in an array for piercing the stratum corneum. The microprojection member can be formed by etching or punching a plurality of microprojections from a thin sheet and folding or bending the microprojections out of the plane of the sheet to form a configuration, such as that shown in FIG. 2. The microprojection member can also be formed in other known manners, such as by forming one or more strips having microprojections along an edge of each of the strip(s) as disclosed in U.S. Pat. No. 6,050,988, which is hereby incorporated by reference in its entirety.

The term “frequency assisted”, as used herein, generally refers to the delivery of a therapeutic agent (charged, uncharged, or mixtures thereof), particularly a vaccine, through a body surface (such as skin, mucous membrane, or nails) wherein the delivery is at least partially induced or aided by the application of high frequencies that produce oscillations in a microprojection member and/or microprojection array thereof.

As indicated above, the present invention generally comprises (i) a microprojection member (or system) having a plurality of microprojections (or array thereof) that are adapted to pierce through the stratum corneum into the underlying epidermis layer, or epidermis and dermis layers and (ii) a oscillation inducing device for transdermal delivery of biologically active agents.

In one embodiment, the microprojections have a coating thereon that contains at least one biologically active agent, such as a vaccine. Upon piercing the stratum corneum layer of the skin, the agent-containing coating is dissolved by body fluid (intracellular fluids and extracellular fluids such as interstitial fluid) and released into the skin (i.e., bolus delivery) for systemic therapy. As discussed in detail herein, after application of the microprojection member, the microprojection member is subjected to high frequency oscillations via the oscillation inducing device to, among other things, enhance agent flux.

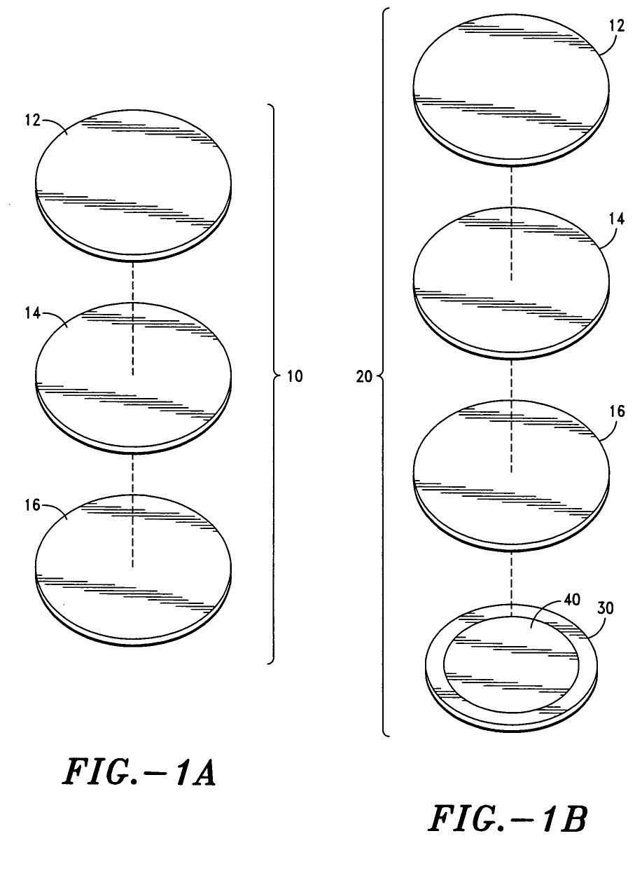

Referring now to FIG. 1A there is shown a schematic illustration of an exemplary oscillation inducing device that can be used in accordance with the present invention. As illustrated in FIG. 1A, the oscillation inducing device 10 generally includes a backing member 12, an energy source 14, such as a thin film capacitor system (and associated circuitry) and a thin film oscillator 16, such as a ceramic piezoelectric oscillator. Preferably, the backing member 12 includes a skin adhesive ring or tabs (not shown) to facilitate adherence of the oscillation device 10 on the patient's skin.

In a preferred embodiment, the oscillation inducing device 10, 20 provides high frequency vibrations in the range of 200 Hz-100 KHz.

Preferably, the oscillation inducing device 10, 20 produces substantially uniaxial oscillations, in a direction longitudinal with the microprojections, in the associated microprojection member (e.g., 30) in the range of approximately 10-400 μm.

In an alternative embodiment, the oscillation inducing device 10, 20 produces substantially transversal oscillations of the associated microprojection member (e.g., 30). Such transversal oscillations can facilitate the cutting action of the microprojections.

In another alternative embodiment, the oscillation inducing device 10, 20 produces substantially circular oscillations of the associated microprojection member (e.g., 30). Such circular oscillations can facilitate the cutting action of the microprojections.

In an alternative embodiment, the system further comprises an ultrasonic device to facilitate delivery of the biologically active agent. Preferably, the ultrasonic device provides sound waves having a frequency in the range of approximately 20 kHz-10 MHz.

As will be appreciated by one having ordinary skill in the art, various oscillation inducing devices can be employed within the scope of the invention to induce the high frequency oscillations in the microprojection member (e.g., 30).

According to the invention, the oscillation inducing device 10, 20 can be employed with various microprojection members and systems to enhance the agent flux. Referring now to FIG. 2, there is shown one embodiment of a microprojection member 30 for use with the present invention. As illustrated in FIG. 2, the microprojection member 30 includes a microprojection array 32 having a plurality of microprojections 34. The microprojections 34 preferably extend at substantially a 90° angle from the sheet 36, which in the noted embodiment includes openings 38.

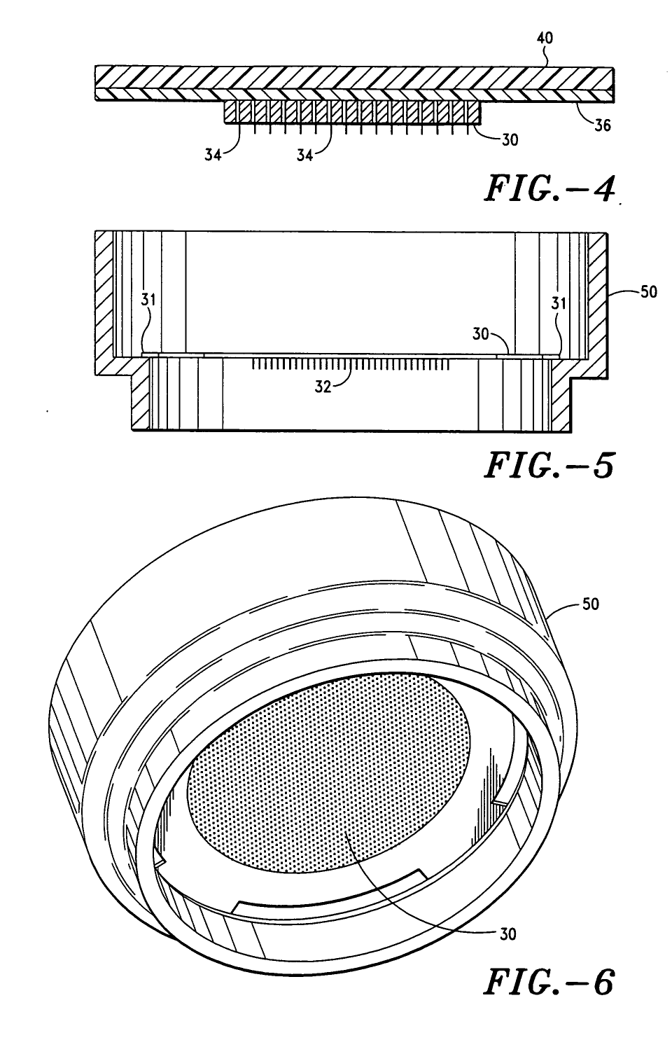

According to the invention, the sheet 36 may be incorporated into a delivery patch, including a backing 40 for the sheet 36, and may additionally include adhesive 16 for adhering the patch to the skin (see FIG. 4). In this embodiment, the microprojections 34 are formed by etching or punching a plurality of microprojections 34 from a thin metal sheet 36 and bending the microprojections 34 out of the plane of the sheet 36.

In one embodiment of the invention, the microprojection member 30 has a microprojection density of at least approximately 10 microprojections/cm2, more preferably, in the range of at least approximately 200-2000 microprojections/cm2. Preferably, the number of openings per unit area through which the agent passes is at least approximately 10 openings/cm2 and less than about 2000 openings/cm2.

As indicated, the microprojections 34 preferably have a projection length less than 1000 microns. In one embodiment, the microprojections 34 have a projection length of less than 500 microns, more preferably, less than 250 microns. The microprojections 34 also preferably have a width and thickness of about 5 to 50 microns.

The microprojection member 30 can be manufactured from various metals, such as stainless steel, titanium, nickel titanium alloys, or similar biocompatible materials, such as polymeric materials. Preferably, the microprojection member 30 is manufactured out of titanium.

According to the invention, the microprojection member 30 can also be constructed out of a non-conductive material, such as a polymer. Alternatively, the microprojection member can be coated with a non-conductive material, such as Parylene®.

Microprojection members that can be employed with the present invention include, but are not limited to, the members disclosed in U.S. Pat. Nos. 6,083,196, 6,050,988 and 6,091,975, which are incorporated by reference herein in their entirety.

Other microprojection members that can be employed with the present invention include members formed by etching silicon using silicon chip etching techniques or by molding plastic using etched micro-molds, such as the members disclosed U.S. Pat. No. 5,879,326, which is incorporated by reference herein in its entirety.

According to the invention, the biologically active agent to be delivered can be contained in the hydrogel formulation disposed in a gel pack reservoir (discussed in detail below), contained in a biocompatible coating that is disposed on the microprojection member 30 or contained in both the hydrogel formulation and the biocompatible coating.

Referring now to FIG. 3, there is shown a microprojection member 30 having microprojections 34 that include a biocompatible coating 35. According to the invention, the coating 35 can partially or completely cover each microprojection 34. For example, the coating 35 can be in a dry pattern coating on the microprojections 34. The coating 35 can also be applied before or after the microprojections 34 are formed.

According to the invention, the coating 35 can be applied to the microprojections 34 by a variety of known methods. Preferably, the coating is only applied to those portions the microprojection member 30 or microprojections 34 that pierce the skin (e.g., tips 39).

One such coating method comprises dip-coating. Dip-coating can be described as a means to coat the microprojections by partially or totally immersing the microprojections 34 into a coating solution. By use of a partial immersion technique, it is possible to limit the coating 35 to only the tips 39 of the microprojections 34.

A further coating method comprises roller coating, which employs a roller coating mechanism that similarly limits the coating 35 to the tips 39 of the microprojections 34. The roller coating method is disclosed in U.S. application Ser. No. 10/099,604 (Pub. No. 2002/0132054), which is incorporated by reference herein in its entirety.

As discussed in detail in the noted application, the disclosed roller coating method provides a smooth coating that is not easily dislodged from the microprojections 34 during skin piercing. The smooth cross-section of the microprojection tip coating is further illustrated in FIG. 3A.

According to the invention, the microprojections 34 can further include means adapted to receive and/or enhance the volume of the coating 35, such as apertures (not shown), grooves (not shown), surface irregularities (not shown) or similar modifications, wherein the means provides increased surface area upon which a greater amount of coating can be deposited.

A further coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 10 and then dried.

Pattern coating can also be employed to coat the microprojections 34. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

As indicated, according to one embodiment of the invention, the coating formulations applied to the microprojection member 30 to form solid coatings can comprise aqueous and non-aqueous formulations having at least one biologically active agent. According to the invention, the biologically active agent can be dissolved within a biocompatible carrier or suspended within the carrier.

According to the invention, the coating formulations preferably include at least one wetting agent. As is well known in the art, wetting agents can generally be described as amphiphilic molecules. When a solution containing the wetting agent is applied to a hydrophobic substrate, the hydrophobic groups of the molecule bind to the hydrophobic substrate, while the hydrophilic portion of the molecule stays in contact with water. As a result, the hydrophobic surface of the substrate is not coated with hydrophobic groups of the wetting agent, making it susceptible to wetting by the solvent. Wetting agents include surfactants as well as polymers presenting amphiphillic properties.

In one embodiment of the invention, the coating formulations include at least one surfactant. According to the invention, the surfactant(s) can be zwitterionic, amphoteric, cationic, anionic, or nonionic. Examples of surfactants include, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laurate, and alkoxylated alcohols such as laureth-4. Most preferred surfactants include Tween 20, Tween 80, and SDS.

Preferably, the concentration of the surfactant is in the range of approximately 0.001-2 wt. % of the coating solution formulation.

In a further embodiment of the invention, the coating formulations include at least one polymeric material or polymer that has amphiphilic properties. Examples of the noted polymers include, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxyethylcellulose (EHEC), as well as pluronics.

In one embodiment of the invention, the concentration of the polymer presenting amphiphilic properties is preferably in the range of approximately 0.01-20 wt. %, more preferably, in the range of approximately 0.03-10 wt. % of the coating formulation. Even more preferably, the concentration of the wetting agent is in the range of approximately 0.1-5 wt. % of the coating formulation.

As will be appreciated by one having ordinary skill in the art, the noted wetting agents can be used separately or in combinations.

According to the invention, the coating formulations can further include a hydrophilic polymer. Preferably the hydrophilic polymer is selected from the following group: poly(vinyl alcohol), poly(ethylene oxide), poly(2-hydroxyethylmethacrylate), poly(n-vinyl pyrolidone), polyethylene glycol and mixtures thereof, and like polymers. As is well known in the art, the noted polymers increase viscosity.

The concentration of the hydrophilic polymer in the coating formulation is preferably in the range of approximately 0.01-20 wt. %, more preferably, in the range of approximately 0.03-10 wt. % of the coating formulation. Even more preferably, the concentration of the wetting agent is in the range of approximately 0.1-5 wt. % of the coating formulation.

According to the invention, the coating formulations can further include a biocompatible carrier such as those disclosed in Co-Pending U.S. application Ser. No. 10/127,108, which is incorporated by reference herein in its entirety. Examples of biocompatible carriers include human albumin, bioengineered human albumin, polyglutamic acid, polyaspartic acid, polyhistidine, pentosan polysulfate, polyamino acids, sucrose, trehalose, melezitose, raffinose and stachyose.

The concentration of the biocompatible carrier in the coating formulation is preferably in the range of approximately 2-70 wt. %, more preferably, in the range of approximately 5-50 wt. % of the coating formulation. Even more preferably, the concentration of the wetting agent is in the range of approximately 10-40 wt. % of the coating formulation.

The coatings of the invention can further include a vasoconstrictor such as those disclosed in Co-Pending U.S. application Ser. Nos. 10/674,626 and 60/514,433, which are incorporated by reference herein in their entirety. As set forth in the noted Co-Pending Applications, the vasoconstrictor is used to control bleeding during and after application on the microprojection member. Preferred vasoconstrictors include, but are not limited to, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating.

In yet another embodiment of the invention, the coating formulations include at least one “pathway patency modulator”, such as those disclosed in Co-Pending U.S. application Ser. No. 09/950,436, which is incorporated by reference herein in its entirety. As set forth in the noted Co-Pending Application, the pathway patency modulators prevent or diminish the skin's natural healing processes thereby preventing the closure of the pathways or microslits formed in the stratum corneum by the microprojection member array. Examples of pathway patency modulators include, without limitation, osmotic agents (e.g., sodium chloride), and zwitterionic compounds (e.g., amino acids).

The term “pathway patency modulator”, as defined in the Co-Pending Application, further includes anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In certain embodiments of the invention, the viscosity and stability of the biologically active agent containing coating formulation is enhanced by adding low volatility counterions. In one embodiment, the agent has a positive charge at the formulation pH and the viscosity-enhancing counterion comprises an acid having at least two acidic pKas. Suitable acids include maleic acid, malic acid, malonic acid, tartaric acid, adipic acid, citraconic acid, fumaric acid, glutaric acid, itaconic acid, meglutol, mesaconic acid, succinic acid, citramalic acid, tartronic acid, citric acid, tricarballylic acid, ethylenediaminetetraacetic acid, aspartic acid, glutamic acid, carbonic acid, sulfuric acid, and phosphoric acid.

Another preferred embodiment is directed to a viscosity-enhancing mixture of counterions wherein the agent has a positive charge at the formulation pH and at least one of the counterions is an acid having at least two acidic pKas. The other counterion is an acid with one or more pKas. Examples of suitable acids include hydrochloric acid, hydrobromic acid, nitric acid, sulfuric acid, maleic acid, phosphoric acid, benzene sulfonic acid, methane sulfonic acid, citric acid, succinic acid, glycolic acid, gluconic acid, glucuronic acid, lactic acid, malic acid, pyruvic acid, tartaric acid, tartronic acid, fumaric acid, acetic acid, propionic acid, pentanoic acid, carbonic acid, malonic acid, adipic acid, citraconic acid, levulinic acid, glutaric acid, itaconic acid, meglutol, mesaconic acid, citramalic acid, citric acid, aspartic acid, glutamic acid, tricarballylic acid and ethylenediaminetetraacetic acid.

Generally, in the noted embodiments of the invention, the amount of counterion should neutralize the charge of the antigenic agent. In such embodiments, the counterion or the mixture of counterion is present in amounts necessary to neutralize the charge present on the agent at the pH of the formulation. Excess of counterion (as the free acid or as a salt) can be added to the formulation in order to control pH and to provide adequate buffering capacity.

In one preferred embodiment, the agent has a positive charge and the counterion is a viscosity-enhancing mixture of counterions chosen from the group of citric acid, tartaric acid, malic acid, hydrochloric acid, glycolic acid, and acetic acid. Preferably, counterions are added to the formulation to achieve a viscosity in the range of about 20-200 cp.

In a preferred embodiment, the viscosity-enhancing counterion is an acidic counterion such as a low volatility weak acid. Low volatility weak acid counterions present at least one acidic pKa and a melting point higher than about 50° C. or a boiling point higher than about 170° C. at Pₐₜₘ. Examples of such acids include citric acid, succinic acid, glycolic acid, gluconic acid, glucuronic acid, lactic acid, malic acid, pyruvic acid, tartaric acid, tartronic acid, and fumaric acid.

In another preferred embodiment the counterion is a strong acid. Strong acids can be defined as presenting at least one pKa lower than about 2. Examples of such acids include hydrochloric acid, hydrobromic acid, nitric acid, sulfonic acid, sulfuric acid, maleic acid, phosphoric acid, benzene sulfonic acid and methane sulfonic acid.

Another preferred embodiment is directed to a mixture of counterions wherein at least one of the counterion is a strong acid and at least one of the counterion is a low volatility weak acid.

Another preferred embodiment is directed to a mixture of counterions wherein at least one of the counterion is a strong acid and at least one of the counterion is a weak acid with high volatility. Volatile weak acid counterions present at least one pKa higher than about 2 and a melting point lower than about 50° C. or a boiling point lower than about 170° C. at Pₐₜₘ. Examples of such acids include acetic acid, propionic acid, pentanoic acid and the like.

The acidic counterion is present in amounts necessary to neutralize the positive charge present on the agent at the pH of the formulation. Excess of counterion (as the free acid or as a salt) can be added to the formulation in order to control pH and to provide adequate buffering capacity.

In yet other embodiments of the invention, particularly where the antigenic agent has a negative charge, the coating formulation further comprises a low volatility basic counter ion.

In a preferred embodiment, the coating formulation comprises a low volatility weak base counterion. Low volatility weak bases present at least one basic pKa and a melting point higher than about 50° C. or a boiling point higher than about 170° C. at Pₐᵣₘ. Examples of such bases include monoethanolomine, diethanolamine, triethanolamine, tromethamine, methylglucamine, and glucosamine.

In another embodiment, the low volatility counterion comprises a basic zwitterion presenting at least one acidic pKa, and at least two basic pKa's, wherein the number of basic pKa's is greater than the number of acidic pkA's. Examples of such compounds include histidine, lysine, and arginine.

In yet other embodiments, the low volatility counterion comprises a strong base presenting at least one pKa higher than about 12. Examples of such bases include sodium hydroxide, potassium hydroxide, calcium hydroxide, and magnesium hydroxide.

Other preferred embodiments comprise a mixture of basic counterions comprising a strong base and a weak base with low volatility. Alternatively, suitable counterions include a strong base and a weak base with high volatility. High volatility bases present at least one basic pKa lower than about 12 and a melting point lower than about 50° C. or a boiling point lower than about 170° C. at Pₐₜₘ. Examples of such bases include ammonia and morpholine.

Preferably, the basic counterion is present in amounts necessary to neutralize the negative charge present on the antigenic agent at the pH of the formulation. Excess of counterion (as the free base or as a salt) can be added to the formulation in order to control pH and to provide adequate buffering capacity.

Further discussion regarding the use of low volatility counterions can be found in U.S. Patent Application Ser. No. 60/484,020, filed Jun. 30, 2003 and 60/484,020, filed Jun. 30, 2003; the disclosures of which are incorporated by reference herein in their entirety.

According to the invention, the coating formulations can also include a non-aqueous solvent, such as ethanol, chloroform, ether, propylene glycol, polyethylene glycol and the like, dyes, pigments, inert fillers, permeation enhancers, excipients, and other conventional components of pharmaceutical products or transdermal devices known in the art.

Other known formulation adjuvants can also be added to the coating formulations as long as they do not adversely affect the necessary solubility and viscosity characteristics of the coating formulation and the physical integrity of the dried coating.

Preferably, the coating formulations have a viscosity less than approximately 500 centipoise and greater than 3 centipoise in order to effectively coat each microprojection 10. More preferably, the coating formulations have a viscosity in the range of approximately 3-200 centipoise.

According to the invention, the desired coating thickness is dependent upon the density of the microprojections per unit area of the sheet and the viscosity and concentration of the coating composition as well as the coating method chosen. Preferably, the coating thickness is less than 50 microns.

In one embodiment, the coating thickness is less than 25 microns, more preferably, less than 10 microns as measured from the microprojection surface. Even more preferably, the coating thickness is in the range of approximately 1 to 10 microns.

In all cases, after a coating has been applied, the coating formulation is dried onto the microprojections 10 by various means. In a preferred embodiment of the invention, the coated member 5 is dried in ambient room conditions. However, various temperatures and humidity levels can be used to dry the coating formulation onto the microprojections. Additionally, the coated member 5 can be heated, lyophilized, freeze dried or similar techniques used to remove the water from the coating.

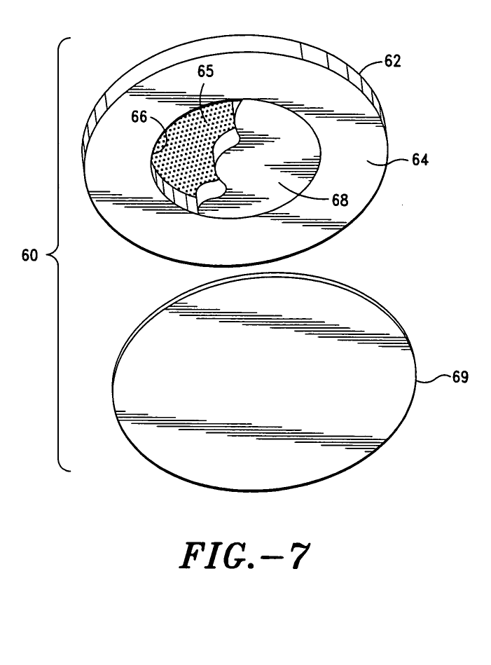

Referring now to FIGS. 5 and 6, for storage and application (in accordance with one embodiment of the invention), the microprojection member 30 is preferably suspended in a retainer ring 50 by adhesive tabs 31, as described in detail in Co-Pending U.S. application Ser. No. 09/976,762 (Pub. No. 2002/0091357), which is incorporated by reference herein in its entirety.

After placement of the microprojection member 30 in the retainer ring 50, the microprojection member 30 is applied to the patient's skin. Preferably, the microprojection member 30 is applied to the skin using an impact applicator, such as disclosed in Co-Pending U.S. application Ser. No. 09/976,798, which is incorporated by reference herein in its entirety.

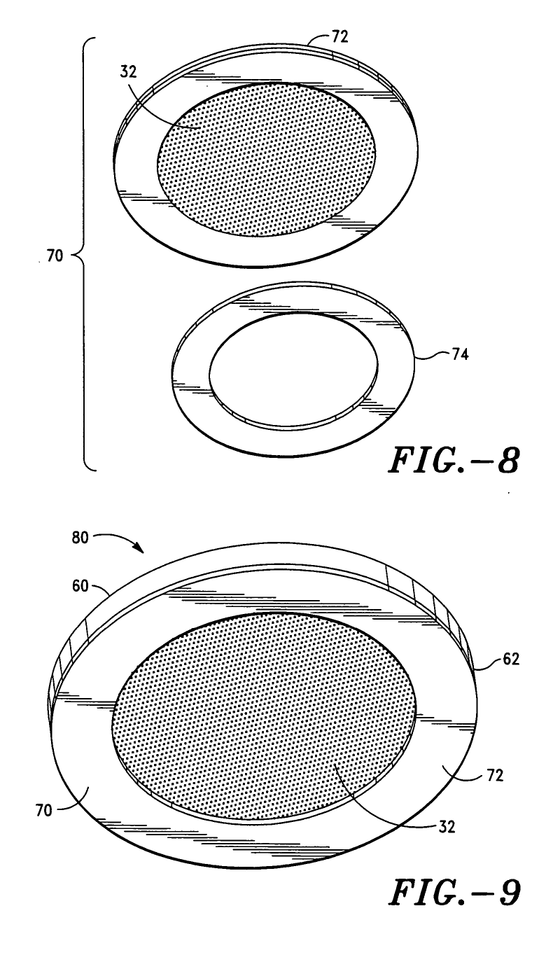

Referring now to FIGS. 7 and 8, there is shown a further microprojection (or delivery) system that can be employed within the scope of the present invention. As illustrated in FIGS. 7 and 8, the system 60 includes a gel pack 62 and a microprojection assembly 70, having a microprojection member, such as the microprojection member 30 shown in FIG. 2.

According to the invention, the gel pack 62 includes a housing or ring 64 having a centrally disposed reservoir or opening 66 that is adapted to receive a predetermined amount of a hydrogel formulation 68 therein. As illustrated in FIG. 7, the ring 64 further includes a backing member 65 that is disposed on the outer planar surface of the ring 64. Preferably, the backing member 65 is impermeable to the hydrogel formulation.

In a preferred embodiment, the gel pack 60 further includes a strippable release liner 69 that is adhered to the outer surface of the gel pack ring 64 via a conventional adhesive. As described in detail below, the release liner 69 is removed prior to application of the gel pack 60 to the applied (or engaged) microprojection assembly 70.

Referring now to FIG. 8, the microprojection assembly 70 includes a backing membrane ring 72 and a similar microprojection array 32. The microprojection assembly further includes a skin adhesive ring 74.

Further details of the illustrated gel pack 60 and microprojection assembly 70, as well as additional embodiments thereof that can be employed within the scope of the present invention are set forth in Co-Pending Application No. 60/514,387, which is incorporated by reference herein in its entirety.

As indicated above, in at least one embodiment of the invention, the hydrogel formulation contains at least one biologically active agent, such as a vaccine. In an alternative embodiment of the invention, the hydrogel formulation is devoid of a biologically active agent and, hence, is merely a hydration mechanism.

According to the invention, when the hydrogel formulation is devoid of a biologically active agent, the active agent is either coated on the microprojection array 32, as described above, or contained in a solid film, such as disclosed in PCT Pub. No. WO 98/28037, which is similarly incorporated by reference herein in its entirety, on the skin side of the microprojection array 32, such as disclosed in the noted Co-Pending Application No. 60/514,387 or the top surface of the array 32.

As discussed in detail in the Co-Pending Application, the solid film is typically made by casting a liquid formulation consisting of the biologically active agent, a polymeric material, such as hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), or pluronics, a plasticising agent, such as glycerol, propylene glycol, or polyethylene glycol, a surfactant, such as Tween 20 or Tween 80, and a volatile solvent, such as water, isopropanol, or ethanol. Following casting and subsequent evaporation of the solvent, a solid film is produced.

Preferably, the hydrogel formulations of the invention comprise water-based hydrogels. Hydrogels are preferred formulations because of their high water content and biocompatibility.

As is well known in the art, hydrogels are macromolecular polymeric networks that are swollen in water. Examples of suitable polymeric networks include, without limitation, hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics. The most preferred polymeric materials are cellulose derivatives. These polymers can be obtained in various grades presenting different average molecular weight and therefore exhibit different rheological properties.

Preferably, the concentration of the polymeric material is in the range of approximately 0.5-40 wt. % of the hydrogel formulation.

The hydrogel formulations of the invention preferably have sufficient surface activity to insure that the formulations exhibit adequate wetting characteristics, which are important for establishing optimum contact between the formulation and the microprojection array 32 and skin and, optionally, the solid film.

According to the invention, adequate wetting properties are achieved by incorporating a wetting agent in the hydrogel formulation. Optionally, a wetting agent can also be incorporated in the solid film.

Preferably, the wetting agents include at least one surfactant. According to the invention, the surfactant(s) can be zwitterionic, amphoteric, cationic, anionic, or nonionic. Examples of surfactants include, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laurate, and alkoxylated alcohols such as laureth-4. Most preferred surfactants include Tween 20, Tween 80, and SDS.

Preferably, the wetting agents also include polymeric materials or polymers having amphiphilic properties. Examples of the noted polymers include, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxyethylcellulose (EHEC), as well as pluronics.

Preferably, the concentration of the surfactant is in the range of approximately 0.001-2 wt. % of the hydrogel formulation. The concentration of the polymer that exhibits amphiphilic properties is preferably in the range of approximately 0.5-40 wt. % of the hydrogel formulation.

As will be appreciated by one having ordinary skill in the art, the noted wetting agents can be used separately or in combinations.

According to the invention, the hydrogel formulations can similarly include at least one pathway patency modulator, such as those disclosed in Co-Pending U.S. application Ser. No. 09/950,436. As indicated above, the pathway patency modulator can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextran sulfate sodium, and EDTA.

The hydrogel formulation can further include at least one vasoconstrictor. Suitable vasoconstrictors include, without limitation, epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline, and the mixtures thereof.

According to the invention, the hydrogel formulations can also include a non-aqueous solvent, such as ethanol, propylene glycol, polyethylene glycol and the like, dyes, pigments, inert fillers, permeation enhancers, excipients, and other conventional components of pharmaceutical products or transdermal devices known in the art.

The hydrogel formulations of the invention exhibit adequate viscosity so that the formulation can be contained in the gel pack 60, keeps its integrity during the application process, and is fluid enough so that it can flow through the microprojection assembly openings and into the skin pathways.

For hydrogel formulations that exhibit Newtonian properties, the viscosity of the hydrogel formulation is preferably in the range of approximately 2-30 Poises (P), as measured at 25° C. For shear-thinning hydrogel formulations, the viscosity, as measured at 25° C., is preferably in the range of 1.5-30 P or 0.5 and 10 P, at shear rates of 667/s and 2667/s, respectively. For dilatant formulations, the viscosity, as measured at 25° C., is preferably in the range of approximately 1.5-30 P, at a shear rate of 667/s.

As indicated, in at least one embodiment of the invention, the hydrogel formulation contains at least one vaccine. Preferably, the vaccine comprises one of the aforementioned vaccines.

According to the invention, when the hydrogel formulation contains one of the aforementioned vaccines, the vaccine can be present at a concentration in excess of saturation or below saturation. The amount of a vaccine employed in the microprojection system will be that amount necessary to deliver a therapeutically effective amount of the vaccine to achieve the desired result. In practice, this will vary widely depending upon the particular vaccine, the site of delivery, the severity of the condition, and the desired therapeutic effect. Thus, it is not practical to define a particular range for the therapeutically effective amount of a vaccine incorporated into the method.

In one embodiment of the invention, the concentration of the vaccine is in the range of at least 1-40 wt. % of the hydrogel formulation.

According to one embodiment of the invention, for storage and application, the microprojection assembly is similarly preferably suspended in the retainer 50 shown in FIGS. 5 and 6. After placement of the microprojection assembly 70 in the retainer 50, the microprojection assembly 70 is applied to the patient's skin. Preferably, the microprojection assembly 70 is similarly applied to the skin using an impact applicator, such as disclosed in Co-Pending U.S. application Ser. No. 09/976,798.

After application of the microprojection assembly 70, the release liner 69 is removed from the gel pack 60. The gel pack 60 is then placed on the microprojection assembly 70, whereby the hydrogel formulation 68 is released from the gel pack 60 through the openings 38 in the microprojection array 32, passes through the microslits in the stratum corneum formed by the microprojections 34, migrates down the outer surfaces of the microprojections 34 and through the stratum corneum to achieve local or systemic therapy.

Referring now to FIG. 9, there is shown another embodiment of a microprojection system 80 that can be employed within the scope of the present invention. As illustrated in FIG. 9, the system comprises an integrated unit comprising the microprojection member 70 and gel pack 60 described above and shown in FIGS. 7 and 8.

In accordance with one embodiment of the invention, the method for delivering a biologically active agent (contained in the hydrogel formulation or contained in the biocompatible coating on the microprojection member or both) comprises the following steps: the coated microprojection member (e.g., 70) is initially applied to the patient's skin via an actuator wherein the microprojections 34 pierce the stratum corneum. The oscillation inducing device 10 is then placed on the applied microprojection member and a frequency in the range of 200 Hz-100 kHz is applied.

In an alternative embodiment, wherein the microprojection member is incorporated into the oscillation inducing device 20, the oscillation inducing device 20 is placed on the patient's skin proximate a delivery site, whereby the microprojections pierce the stratum corneum and a frequency in the range of 200 Hz-100 kHz is applied.

Preferably, the microprojections 34 oscillate in the range of approximately 10-400 μm, more preferably.

In one embodiment of the invention, the microprojection member includes a microprojection array 34 having a biocompatible coating disposed thereon that includes at least one biologically active agent, as illustrated in FIG. 3.

In a further embodiment, the microprojection member comprises a microprojection array/gel pack assembly 80, as illustrated in FIG. 9, wherein the gel pack 60 includes an agent-containing hydrogel formulation.

In an alternative embodiment, the biologically active agent is contained in hydrogel formulation in the gel pack 60 and in a biocompatible coating applied to the microprojection member.

From the foregoing description, one of ordinary skill in the art can easily ascertain that the present invention, among other things, provides an effective and efficient means for enhancing the transdermal flux of a biologically active agent into and through the stratum corneum of a patient.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A delivery system for delivering a biologically active agent to a subject, comprising:
a microprojection member having a plurality of stratum corneum-piercing microprojections;
a formulation having said biologically active agent; and
an oscillation inducing device that is adapted to cooperate with the microprojection member to produce high frequency oscillations.

2. The system of claim 1, wherein said oscillation inducing device produces substantially uniaxial oscillations.

3. The system of claim 2, wherein said oscillation inducing device produces oscillations of said microprojection member in the range of approximately 10-400 μm.

4. The system of claim 1, wherein said oscillation inducing device produces substantially transversal oscillations.

5. The system of claim 1, wherein said oscillation inducing device produces substantially circular oscillations.

6. The system of claim 1, wherein said oscillation inducing device provides high frequency vibrations in the range of approximately 200 Hz-100 kHz.

7. The system of claim 1, wherein said oscillation inducing device comprises an ultrasonic device adapted to apply ultrasonic energy to said subject.

8. The system of claim 7, wherein said ultrasonic device generates sound waves having a frequency in the range of approximately 20 kHz to 10 MHz.

9. The system of claim 1, wherein said microprojection member has a microprojection density of at least approximately 10 microprojections/cm².

10. The system of claim 1, wherein said microprojection member has a microprojection density in the range of at least approximately 200-2000 microprojections/cm².

11. The system of claim 1, wherein said microprojections are adapted to pierce through the stratum corneum to a depth of less than about 500 microns.

12. The system of claim 1, wherein said biologically active agent comprises an immunologically active agent selected from the group consisting of proteins, polysaccharide conjugates, oligosaccharides, lipoproteins, subunit vaccines, Bordetella pertussis (recombinant PT accince—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7, Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), Vibrio cholerae (conjugate lipopolysaccharide), whole virus, bacteria, weakened or killed viruses, cytomegalo virus, hepatitis B virus, hepatitis C virus, human papillomavirus, rubella virus, varicella zoster, weakened or killed bacteria, bordetella pertussis, clostridium tetani, corynebacterium diptheriae, group A streptococcus, legionella pneumophila, neisseria meningitis, pseudomonas aeruginosa, streptococcus pneumoniae, treponema pallidum, vibrio cholerae, flu vaccines, Lyme disease vaccine, rabies vaccine, measles vaccine, mumps vaccine, chicken pox vaccine, small pox vaccine, hepatitis vaccine, pertussis vaccine, diptheria vaccine, nucleic acids, single-stranded and double-stranded nucleic acids, supercoiled plasmid DNA, linear plasmid DNA, cosmids, bacterial artificial chromosomes (BACs), yeast artificial chromosomes (YACs), mammalian artificial chromosomes, and RNA molecules.

13. The system of claim 12, wherein said formulation includes an immunologically potentiating adjuvant.

14. The system of claim 13, wherein said adjuvant is selected from the group consisting of aluminum phosphate gel, aluminum hydroxide, algal glucan, b-glucan, cholera toxin B subunit, CRL 1005, ABA block polymer with mean values of x=8 and y=205, gamma insulin, linear (unbranched) β-D(2->1) polyfructofuranoxyl-a-D-glucose, Gerbu adjuvant, N-acetylglucosamine-(b 1-4)-N-acetylmuramyl-L-alanyl-D-glutamine (GMDP), dimethyl dioctadecylammonium chloride (DDA), zinc L-proline salt complex (Zn-Pro-8), Imiquimod (1-(2-methypropyl)-1H-imidazo[4,5-c]quinolin-4-amine, ImmTher™, N-acetylglucoaminyl-N-acetylmuramyl-L-Ala-D-isoGlu-L-Ala-glycerol dipalmitate, MTP-PE liposomes, C59H108N6O19PNa-3H20 (MTP), Murametide, Nac-Mur-L-Ala-D-Gln-OCH3, Pleuran, b-glucan, QS-21; S-28463, 4-amino-a, a-dimethyl-1H-imidazo[4,5-c]quinoline-1-ethanol, sclavo peptide, VQGEESNDK·HCl (IL-1b 163-171 peptide), threonyl-MDP (Termurtide™), N-acetyl muramyl-L-threonyl-D-isoglutamine, interleukin 18, IL-2 IL-12, IL-15, DNA oligonucleotides, CpG containing oligonucleotides, gamma interferon, NF kappa B regulatory signaling proteins, heat-shock proteins (HSPs), GTP-GDP, Loxoribine, MPL®), Murapalmitine, and Theramide™.

15. The system of claim 1, wherein said biologically active agent is selected from the group consisting of leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotropin (ACTH), ACTH analogs such as ACTH (1-24), calcitonin, vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N-[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, BNP, VEGF, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and mixtures thereof.

16. The system of claim 1, wherein said formulation comprises a coating disposed on at least one of said microprojections.

17. The system of claim 16, wherein said formulation includes a surfactant.

18. The system of claim 17, wherein said surfactant is selected from the group consisting of sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, sorbitan derivatives, sorbitan laurate, alkoxylated alcohols, and laureth-4.

19. The system of claim 18, wherein said formulation includes an amphiphilic polymer.

20. The system of claim 19, wherein said amphiphilic polymer is selected from the group consisting of cellulose derivatives, hydroxyethylcellulose (HEC), hydroxypropyl-methylcellulose (HPMC), hydroxypropycellulose (HPC), methylcellulose (MC), hydroxyethylmethylcellulose (HEMC), ethylhydroxyethylcellulose (EHEC), and pluronics.

21. The system of claim 16, wherein said formulation includes a hydrophilic polymer.

22. The system of claim 21, wherein said hydrophilic polymer is selected from the group consisting of poly(vinyl alcohol), poly(ethylene oxide), poly(2-hydroxyethylmethacrylate), poly(n-vinyl pyrolidone), polyethylene glycol and mixtures thereof.

23. The system of claim 16, wherein said formulation includes a biocompatible carrier.

24. The system of claim 23, wherein said biocompatible polymer is selected from the group consisting of human albumin, bioengineered human albumin, polyglutamic acid, polyaspartic acid, polyhistidine, pentosan polysulfate, polyamino acids, sucrose, trehalose, melezitose, raffinose and stachyose.

25. The system of claim 16, wherein said formulation includes a vasoconstrictor.

26. The system of claim 25, wherein said vasoconstrictor is selected from the group consisting of epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline.

27. The system of claim 16, wherein said formulation includes a pathway patency modulator.

28. The system of claim 27, wherein said pathway patency modulator is selected from the group consisting of osmotic agents, sodium chloride, zwitterionic compounds, amino acids, anti-inflammatory agents, betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate, prednisolone 21-succinate sodium salt, anticoagulants, citric acid, citrate salts, sodium citrate, dextran sulfate sodium, and EDTA.

29. The system of claim 16, wherein said formulation includes an antioxidant.

30. The system of claim 29, wherein said antioxidant is selected from the group consisting of sodium citrate, citric acid, ethylene-dinitrilo-tetraacetic acid (EDTA), ascorbic acid, methionine, and sodium ascorbate.

31. The system of claim 16, wherein said formulation further includes a low volatility counterion.

32. The system of claim 31, wherein said low volatility counterion is selected from the group consisting of maleic acid, malic acid, malonic acid, tartaric acid, adipic acid, citraconic acid, fumaric acid, glutaric acid, itaconic acid, meglutol, mesaconic acid, succinic acid, citramalic acid, tartronic acid, citric acid, tricarballylic acid, ethylenediaminetetraacetic acid, aspartic acid, glutamic acid, carbonic acid, sulfuric acid, and phosphoric acid, and mixtures thereof.

33. The system of claim 31, wherein said low volatility counterion is selected from the group consisting of monoethanolomine, diethanolamine, triethanolamine, tromethamine, methylglucamine, glucosamine, histidine, lysine, arginine, sodium hydroxide, potassium hydroxide, calcium hydroxide, magnesium hydroxide, ammonia and morpholine, and mixtures thereof.

34. The system of claim 16, wherein said coating has a viscosity less than approximately 500 centipoise and greater than 3 centipoise.

35. The system of claim 16, wherein said coating has a thickness less than approximately 25 microns.

36. The system of claim 1, wherein said formulation comprises a hydrogel.

37. The system of claim 36, wherein said hydrogel comprises a macromolecular polymeric network.

38. The system of claim 37, wherein said macromolecular polymeric network is selected from the group consisting of hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics.

39. The system of claim 36, wherein said formulation includes a surfactant.

40. The system of claim 39, wherein said surfactant is selected from the group consisting of sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, sorbitan derivatives, sorbitan laurate, alkoxylated alcohols, and laureth-4.

41. The system of claim 36, wherein said formulation includes an amphiphilic polymer.

42. The system of claim 41, wherein said amphiphilic polymer is selected from the group consisting of cellulose derivatives, hydroxyethylcellulose (HEC), hydroxypropyl-methylcellulose (HPMC), hydroxypropycellulose (HPC), methylcellulose (MC), hydroxyethylmethylcellulose (HEMC), ethylhydroxyethylcellulose (EHEC), and pluronics.

43. The system of claim 36, wherein said formulation includes a pathway patency modulator.

44. The system of claim 43, wherein said pathway patency modulator is selected from the group consisting of osmotic agents, sodium chloride, zwitterionic compounds, amino acids, anti-inflammatory agents, betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate, prednisolone 21-succinate sodium salt, anticoagulants, citric acid, citrate salts, sodium citrate, dextran sulfate sodium, and EDTA.

45. The system of claim 36, wherein said formulation includes a vasoconstrictor.

46. The system of claim 45, wherein said vasoconstrictor is selected from the group consisting of epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline.

47. A method for transdermally delivering an biologically active agent to a subject, comprising:
providing a system with a microprojection member having a plurality of stratum corneum-piercing microprojections, a formulation having said biologically active agent and an oscillation inducing device that is adapted to cooperate with the microprojection member to produce oscillations;
applying said microprojection member to a desired location on said subject; and
activating said oscillation inducing device to facilitate penetration of said microprojections into said subject.

48. The method of claim 47, wherein said step of activating said oscillation inducing device generates substantially uniaxial oscillations of said microprojections.

49. The method of claim 48, wherein said step of activating said oscillation inducing device generates substantially uniaxial oscillations of said microprojections in the range of approximately 10-400 μm.

50. The method of claim 48, wherein said step of activating said oscillation inducing device generates substantially transversal oscillations of said microprojections.

51. The method of claim 48, wherein said step of activating said oscillation inducing device generates substantially circular oscillations of said microprojections.

52. The method of claim 48, wherein said step of activating said oscillation inducing device generates high frequency vibrations of said microprojections in the range of approximately 200 Hz-100 kHz.

53. The method of claim 47, wherein said oscillation inducing device comprises an ultrasonic device adapted to transmit ultrasonic energy to said microprojections.

54. The method of claim 53, wherein said ultrasonic device generates sound waves having a frequency in the range of approximately 20 kHz to 10 MHz.
